(19) United States
(12) Patent Application Publication
(10) Pub. No.: US 2010/0101579 A1
(43) Pub. Date: Apr. 29, 2010

(54) Portable Medical Gas Delivery System

(76) Inventor: Frank Levy, Fort Meyers, FL (US)

Correspondence Address: ROBERT M. SCHWARTZ, P.A., P.O. BOX 221470, HOLLYWOOD, FL, 33022, US

(21) Appl. No.: 12/652,845

(22) Filed: Jan. 6, 2010

Related U.S. Application Data
(63) Continuation-in-part of application No. 12/210,368, filed on Sep. 15, 2008.
Application No. 12/210,368 is a continuation-in-part of application No. 11/945,674, filed on Nov. 27, 2007, now Pat. No. 7,543,760.
(60) Provisional application No. 60/867,323, filed on Nov. 27, 2006.

(51) Int. Cl. A61M 16/20; A61M 37/00

(52) U.S. Cl. 128/205.24; 604/23

(57) ABSTRACT

An apparatus and method for delivery of medical grade CO2 liquid to CO2 gas in a safe and consistent manner for delivery to a selected reservoir.

### INDEX TO RELATED APPLICATIONS

This application is a continuation-in-part of U.S. patent application Ser. No. 12/210,368 filed Sep. 15, 2008, which is a continuation-in-part of U.S. patent application Ser. No. 11/945,674 filed Nov. 27, 2007, now U.S. Pat. No. 7,543,760, which claims the benefit of U.S. Provisional Patent Application Ser. No. 60/867,323 filed Nov. 27, 2006 the disclosures of which are incorporated herein by reference in their entirety.

### BACKGROUND OF THE INVENTION

The present invention is intended to provide a portable, safe, consistent, and convenient source of medical CO2 gas to health care professionals in hospital or medical office settings where a small volume of CO2 is needed. The device is intended for general use by physicians and is not intended to be used for any specific medical treatment or procedure The present invention is simple to manufacture and use because it does not require large regulators or external power, cumbersome large tanks or impellers for dispensing medical grade CO2.

The portable apparatus of the present invention utilizes a source of compressed gas to produce the desired pressure and airflow for the effective transformation of medical CO2 liquid to medical grade CO2 gas.

### BRIEF SUMMARY OF THE INVENTION

The present invention provides for a novel apparatus for delivering medical grade CO2 gas.

In one embodiment the present invention is an apparatus for producing medical grade CO2 gas.
(a) a compressed gas unit having at least one container of compressed gas, a source of electric power, and the gas regulator valve.

The compressed gas is any suitable compressed gas. Suitable compressed gases may include carbon dioxide, atmospheric air, nitrogen, helium, or mixtures thereof.

The compressed gas is contained in one or more compressed gas containers.

The apparatus has source of electric power that may be delivered by batteries providing between about 3-24 volts.

In a preferred embodiment, the gas regulator valve is an electronically activated solenoid.

Additionally preferred, the gas regulator valve is an electronically activated solenoid controlled by a pressure activation switch or actuator.

The pressure switch activates the solenoid when depressed.

In one embodiment, the present invention is a CO2 delivery system transforming liquid CO2 in gas.

Any compressed gas can be used. Preferably, the compressed gas is selected from compressed ambient air, carbon dioxide, nitrogen, helium, oxygen, or combinations thereof.

In one embodiment, the apparatus of the present invention includes compressed air storage, with a hose or other acceptable transport mechanism to deliver the compressed gas to any reservoir, Angiobag, or any other receptacle.

In one embodiment, a user will utilize two separate units of the apparatus wherein a first unit includes at least one compressed air cylinder and a valve for controlling the release of compressed air from the cylinder. In one embodiment the valve for controlling the release of compressed air is an electronic solenoid.

The present invention also relates to methods of medical treatment.

In one embodiment. The invention is a method for providing medical grade CO2 gas comprising the steps of:
(a) providing a portable CO2 apparatus;
(b) providing said container having an entrance, an exit, and a release means regulating said exit;
(c) initiating an actuator of the apparatus to release CO2;

In medical uses, CO2 is used because it is safer and has fewer complications than air or oxygen in the same uses. CO2 diffuses more naturally in body tissues and is absorbed in the body more rapidly and with fewer side effects.

CO2 is used in de-compartmentalization of tissues, arteries, veins, and nerves, and radiological imaging, cardiac imaging for evaluation of the vascularity of the heart and surrounding tissues, oncology and urology diagnostics

It is used for imaging by infiltrating the tissues, body cavities, and abdomen for better visualization. The CO2 can also expand internal body cavities and tissues enabling better diagnostic techniques.

In a preferred embodiment, the CO2 gas that is expelled from the invention will ultimately be delivered to a reservoir of the physician's choice

The invention can be used for the delivery of CO2 or other gases into a medical ambu bag system that will be delivered to a reservoir of the physician's choice. The ambu bag is a reservoir that is used to house the CO2 that is provided from existing CO2 tanks

The present invention is portable, compact, and electronic for the use in the field for portable medical uses, military field use, and any other use requiring CO2 for its performance, such as; imaging, tissue separation, vascular/vessel compartmentalization, cellulite, stretch marks, facial wrinkles, and dark circles.

The present invention can also be adapted and used for other gases to be dispensed, under the same nature, such as; oxygen, nitrogen, helium, or any other gas needed to be contained in a compact, portable delivery system.

In a preferred embodiment, the present invention is completely battery operated.

The invention can be used to provide CO2 or like gases to any reservoir of the physician's choice, or any container that can house or store the CO2 before using it in medical devices, such as; imaging, differentiation of tissues, arterial/venous/neurological separation, treatment of stretch marks, facial wrinkles and dark circles.

The invention can deliver CO2 from an adjustable port with control of the pressure in PSI (pounds per square inch) from about 0 PSI to about 120 PSI.

Previous methods utilizing large CO2 tanks and regulators are dangerous because of the risk of a seal, valve, or part malfunction causing a projectile in a medical setting. The present invention is safer as it eliminates this possibility of malfunction.

The invention requires very little space to store, as opposed to the cumbersome existing tank systems and is much easier to use, with a push button actuator to initiate operation.

The present invention is much less expensive then current CO2 tank systems.

Acquisition of the CO2 in the present invention now requires only cartridges which can be delivered in a small box. The current tanks require filling at a filling station which involves the transport of a large quantity of CO2 which could also result in an explosion in the event of a motor vehicle crash.

### BRIEF DESCRIPTION OF THE DRAWINGS

FIG. 1 is a side perspective view of an apparatus including compressed gas cylinders and a solenoid of the present invention.

FIG. 2 is an alternative embodiment including 12 gram and 16 gram CO2 cartridges.

FIG. 3 is an alternative embodiment including a larger compressed gas cylinder connected to a solenoid of the present invention.

FIG. 4 is a front view of the casing housing the invention.

FIG. 5 is a schematic front view of the invention inside the casing.

### DETAILED DESCRIPTION OF THE PREFERRED EMBODIMENTS

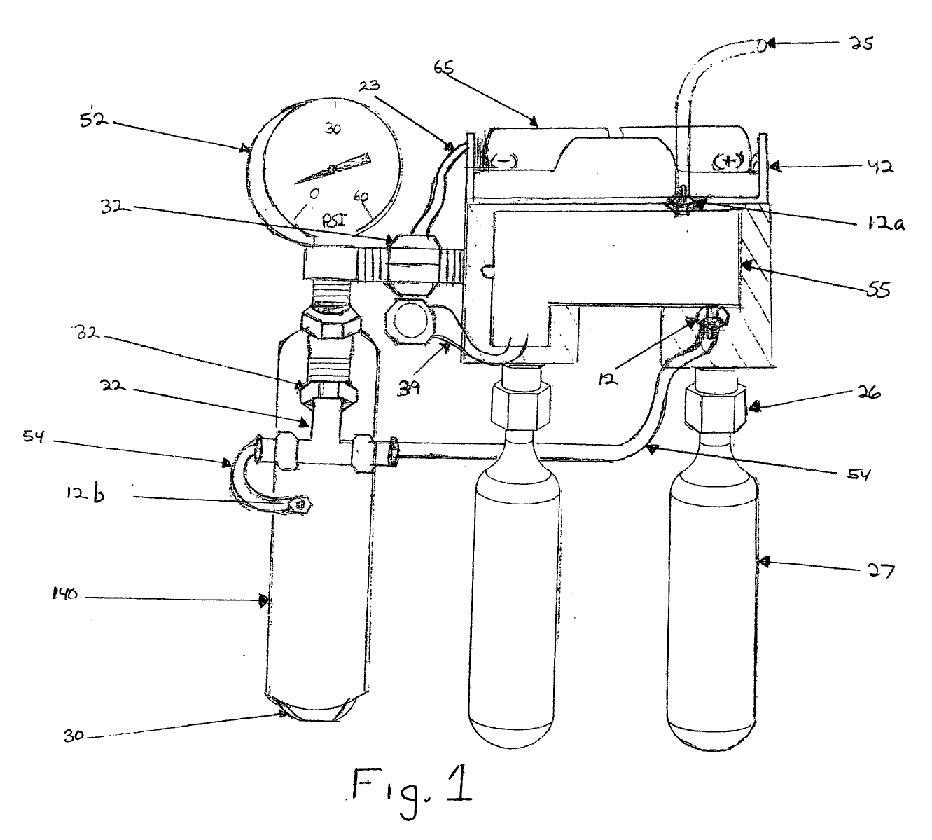

In FIG. 1 compressed gas unit 1 comprises solenoid 55 with at least one compressed gas cylinder. In one embodiment, compressed gas cylinder 27 is 25 g or larger. Compressed gas cylinder 27 is secured into position to unit 1 by means of cylinder cartridge puncture valve 26 and T “puncture” fitting 74.

In a preferred embodiment, cylinder cartridge puncture valve 26 has a mechanism for piercing cylinder 27, as is known, and holding or securing said cylinder in place.

Compressed air enters solenoid 55 from compressed gas cylinder 27 by means of cylinder cartridge puncture valve 26 and T “puncture” fitting 74. Compressed gas unit 1 has at least one battery 65, held in place by battery holder 42, for providing electrical power by which solenoid 55 may be activated and then regulated by pressure activation switch or actuator 37. Battery 65 supplies power to solenoid 55 through battery to switch wire assembly 23, which is secured in place by pressure nut 32. Compressed air unit 1 has an electrical wire 39 for providing necessary electricity to solenoid 55. Unit 1 also comprises black rock regulator 140, which is regulated by secondary regulator adjustment knob 30 when solenoid 55 is activated. Black rock regulator 140 is connected to unit 1 at pressure nut 32 along a threaded mounting. Compressed gas cylinder 27 is secured to unit 1 by cartridge puncture valve 26 as is commonly known. In one embodiment, compressed gas cylinder 27 is a 25 g cylinder. Compressed air leaves black rock regulator 140 by means of a 10/32″ hose port 12b, flows through hose junction 22 by means of ⅛″ pressure hose 54 till reaching the 10/32″ hose port 12b affixed to solenoid 55. From said hose port 12, the compressed air enters solenoid 55. Compressed air unit 1 also has an outlet air port 25, connected to solenoid 55 through intermediate 10/32 hose port 12a, for transporting compressed gas from solenoid 55 in compressed air unit 1. Outlet gas may be monitored with pressure gauge 52. Unit 1 has battery holder 42 for securing battery 65 into position.

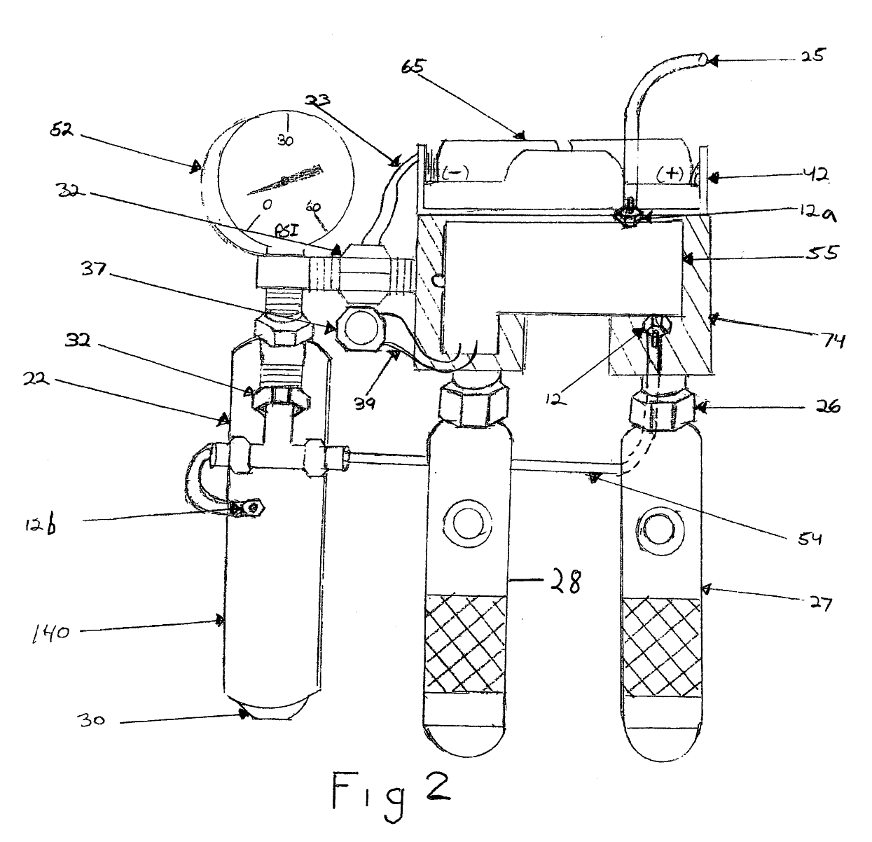

In FIG. 2, one embodiment featuring compressed gas cylinder 28, a 12 g or 16 g compressed gas cylinder, may substitute compressed gas cylinder 27. Housing 74 connects puncture valve 26 with solenoid 55.

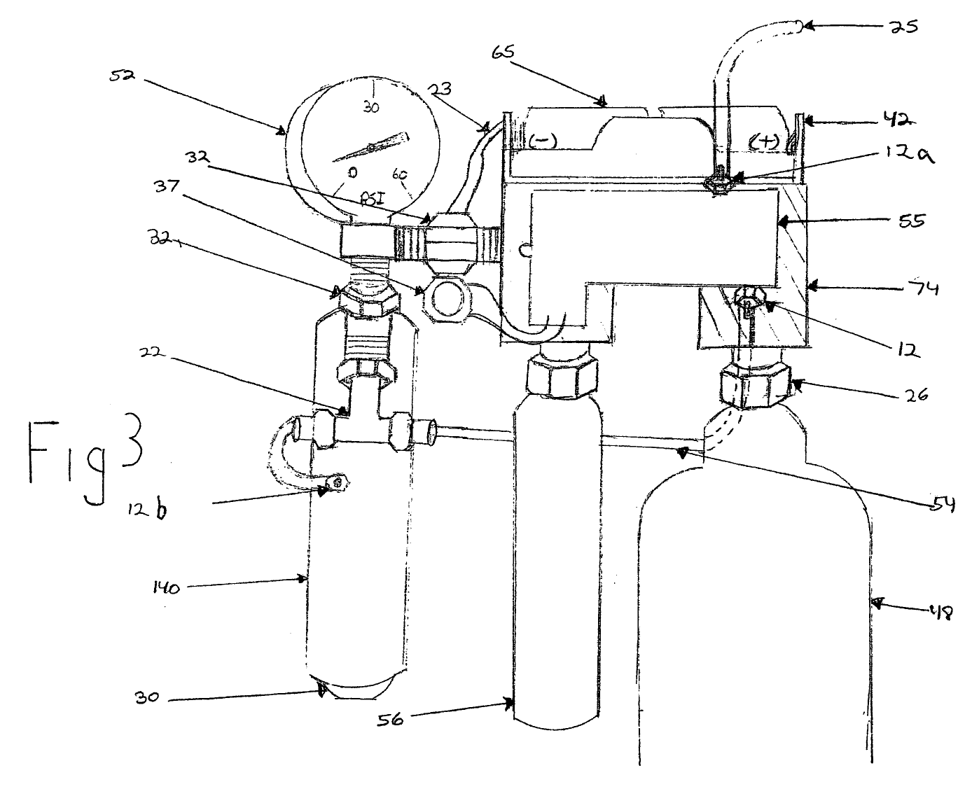

In FIG. 3, another embodiment featuring an accessory tank CO2 delivery system, may substitute compressed gas cylinder 27. Said figure may also substitute a second compressed gas cylinder 27 with expansion chamber 56 used with single compressed gas cylinder 48.

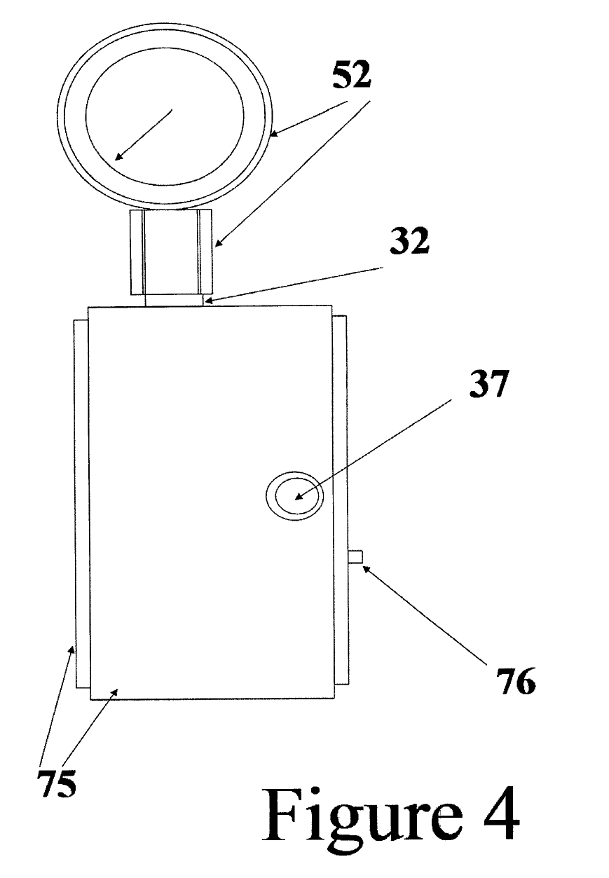

In FIG. 4, shows a front view of the apparatus in a housing 75. Housing 75 has a protruding luer lock fitting 76 accessible by a user outside housing 75.

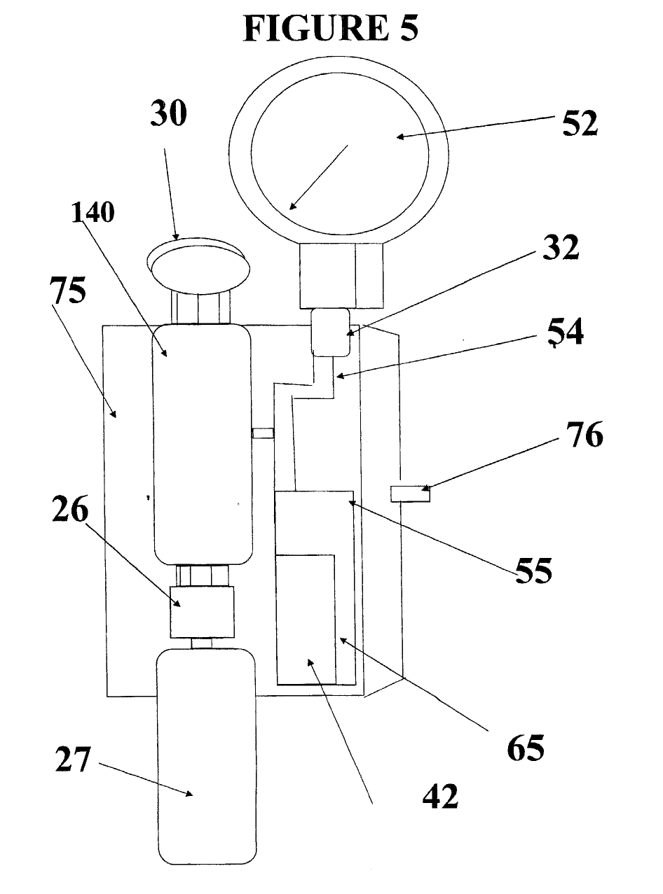

FIG. 5 shows a schematic layout of the components of the present invention as arranged within housing 75.

It is contemplated that the apparatus of the present invention be used in methods and procedures requiring delivery of gas.

CO₂ is useful in the following arterial procedures: abdominal aortography (aneurysm, stenosis), iliac arteriography (stenosis), runoff analysis of the lower extremities (stenosis, occlusion), renal arteriography (stenosis, arteriovenous fistula [AVF], aneurysm, tumor), renal arterial transplantation (stenosis, bleeding, AVF), and visceral arteriography (anatomy, bleeding, AVF, tumor).

CO₂ is useful in the following venous procedures: venography of the upper extremities (stenosis, thrombosis), inferior vena cavography (prior to filter insertion), wedged hepatic venography (visualization of portal vein), direct portography (anatomy, varices), and splenoportography (visualization of portal vein).

CO₂ is in the following interventional procedures: balloon angioplasty (arterial, venous), stent placement (arterial, venous), embolization (renal, hepatic, pelvic, mesenteric), transjugular intrahepatic portacaval shunt creation, and transcatheter biopsy (hepatic, renal).

### EXAMPLES

Angiography is performed by injecting microbubbles of CO2 through a catheter placed in the hepatic artery following conventional hepatic angiography. Vascular findings on US angiography can be classified into four patterns depending on the tumor vascularity relative to the surrounding liver parenchyma: hypervascular, isovascular, hypovascular, and a vascular spot in a hypovascular background.

Improved CT colonography an accurate screening tool for colorectal cancer is performed using a small flexible rectal catheter with automated CO2 delivery improved distension with less post-procedural discomfort.

Carbon dioxide (CO2) gas is used as an alternative contrast to iodinated contrast material. The gas produces negative contrast because of its low atomic number and its low density compared with the surrounding tissues. When injected into a blood vessel, carbon dioxide bubbles displace blood, allowing vascular imaging. Because of the low density of the gas, a digital subtraction angiographic technique is necessary for optimal imaging. The gas bubble can be visible on a standard radiograph and fluoroscopic image.

CO2 insufflation for colonoscopy improves productivity of the endoscopy unit.

Endoscopic thyroid resection—A working space within the neck can be created using CO2 insufflation devices, with both axillary and neck approaches as starting points for dissection.

CO2 unsufflators are used during laparoscopic surgery

Because of the lack of nephrotoxicity and allergic reactions, CO₂ is increasingly used as a contrast agent for diagnostic angiography and vascular interventions in both the arterial and venous circulation.

CO₂ is particularly useful in patients with renal insufficiency or a history of hypersensitivity to iodinated contrast medium.

CO₂ is compressible during injection and expands in the vessel as it exits the catheter.

CO₂ is lighter than blood plasma; therefore, it floats above the blood. When injected into a large vessel such as the aorta or inferior vena cava, CO₂ bubbles flow along the anterior part of the vessel with incomplete blood displacement along the posterior portion.

CO₂ causes no allergic reaction. Because CO₂ is a natural byproduct, it has no likelihood of causing a hypersensitivity reaction. Therefore, the gas is an ideal alternative.

Unlimited amounts of CO₂ can be used for vascular imaging because the gas is effectively eliminated by means of respiration.

CO₂ is particularly useful in patients with compromised cardiac and renal function who are undergoing complex vascular interventions.

Intranasal carbon dioxide is very promising as a safe and effective treatment to provide rapid relief for seasonal allergic rhinitis.

CO2 is used for transient respiratory stimulation; encouragement of deep breathing and coughing to prevent or treat atelectasis; to provide a close-to-physiological atmosphere (mixed with oxygen) for the operation of artificial organs such as the membrane dialyzer (kidney) and the pump oxygenator; and for injection into body cavities during surgical procedures.

Medical asepsis is accomplished by using CO2 on implant devices prior to surgical implantation.

Additionally, the present invention is used in methods requiring the delivery of other gasses such as:

Carbon Dioxide U.S.P.

Medical Air U.S.P.

Helium U.S.P.

Nitrogen N.F.

Nitrous Oxide U.S.P.

Oxygen U.S.P.

In one embodiment, the present invention provides for an apparatus and use in a method whereby delivery of a gas alone is desired. The delivery of gas is independent of systems whereby a gas is delivered as a carrier for medicaments or other materials.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An apparatus for dispensing medical CO2 in any reservoir of the physician's choice:
(a) a portable compressed gas unit having at least one container of compressed gas, a hose for directing said compressed gas, a source of electric power, and a gas regulator valve;
(b) an electronic actuator for initiating and ceasing the flow of said compressed gas;
(c) a medically acceptable directional device for application of said compressed gas;
wherein said apparatus provides compressed gas for medical procedures.

2. The apparatus of claim 1 wherein said compressed gas is a medically acceptable gas.

3. The apparatus of claim 1 wherein said compressed gas is carbon dioxide, atmospheric air, oxygen, or mixtures thereof.

4. The apparatus of claim 1 wherein said compressed gas is supplied with replaceable compressed gas cartridges.

5. The apparatus of claim 1 wherein said source of electric power is delivered to the unit by batteries.

6. The apparatus of claim 1 wherein said actuator is operable as an electronic push button device.

7. The apparatus of claim 1 wherein said actuator is located directly on the apparatus.

8. The apparatus of claim 1 wherein said actuator is operated and positioned remote from the unit.

9. The apparatus of claim 1 wherein said actuator is operable remotely and attached to the directional device.

11. A method for dispensing CO2 in any reservoir of a physician's choice comprising the steps of:
(a) providing a portable CO2 apparatus according to claim 1;
(b) initiating an actuator of the apparatus to release CO2;
(c) delivering CO2 through a directional delivery device.
